(19) United States
(12) Patent Application Publication
(10) Pub. No.: US 2026/0202394 A1
(43) Pub. Date: Jul. 16, 2026

(54) SENSOR DEVICE

(71) Applicant: Oxford Nanopore Technologies PLC, Oxford (GB)

(72) Inventors: David Waterman, Oxford (GB); Thomas Robert Law, Oxford (GB)

(73) Assignee: Oxford Nanopore Technologies PLC, Oxford (GB)

(21) Appl. No.: 19/135,565

(22) PCT Filed: Dec. 1, 2023
(86) PCT No.: PCT/GB2023/053111

§ 371 Date: Jun. 4, 2025

(30) Foreign Application Priority Data
Dec. 5, 2022 (GB) 2218240.6
May 17, 2023 (GB) 2307331.5

(51) Int. Cl. G01N 33/487

(52) U.S. Cl. CPC G01N 33/48721

(57) ABSTRACT

A sensor system comprising a sensor device, a hub component, and a tablet computer. The hub component is connectable to the sensor device, such that the hub component provides an interface for powering the sensor device and transferring data from the sensor device. The hub component further comprises a connection to a computer, such that the hub can deliver data collected from the sensor device to the computer. The height profile dimensions of the sensor device and the hub are similar to the height profile dimension of the tablet computer.

### RELATED APPLICATIONS

This application is a national stage filing under 35 U.S.C. § 371 of international application number PCT/GB2023/053111, filed Dec. 1, 2023, which claims the benefit of United Kingdom application number 2307331.5, filed May 17, 2023, and United Kingdom application number 2218240.6, filed Dec. 5, 2022, each of which is herein incorporated by reference in its entirety.

### TECHNICAL FIELD

The present invention relates to a sensor device for integrating with a tablet computer. More specifically the present invention relates to a sensor device for combining within a folding case of a tablet computer to create an integrated sensor system. Even more specifically, the components of the sensor device are provided such that they can be easily stowed in a consumer case for a tablet computer. Even more specifically, the present invention relates to a nanopore sensor system comprising a nanopore sensor device integrated with a tablet computer and a hub.

### BACKGROUND

Sensor devices are ever increasing in complexity and sensitivity in terms of their functionality and the analytes/substances that they are designed to detect. Coupled with this, there is a desire to increase the usability of the device and user experience such that sensors can be used by unskilled technicians away from laboratory conditions.

Sensor devices comprising an instrument for use with a disposable or reusable test cartridge are well-known, such as point of care diagnostic devices as disclosed by WO21146350, as well as devices such as the Roche Diagnostics CoaguChek™ XS System, https://coaguchek.roche.com/global/en/products/coaguchek-xs-system.html and the Lumira Dx™ diagnostic platform https://www.lumiradx.com/uk-en/. A case in point is the use of nanopore sensors, such as the MinION™, sold by Oxford Nanopore Technologies, https://nanoporetech.com/products/minion to sense interactions with molecular entities, for example polynucleotides. The device comprises a body having a lid that may be opened at one end allowing for attachment of a disposable sensor component on its upper surface. Nanopore sensing is a powerful molecular sensing technique that has been subject to recent developments. Nanopore sensor devices have been developed that comprise an array of nanopore sensing elements, thereby increasing data collection by allowing plural nanopores to sense interactions in parallel, typically from the same sample. Nanopore sensor devices of the type such as described in WO2021111133A2 may be used to analyse the sequence of a polymer to be identified during the analysis of a test sample.

### SUMMARY OF THE INVENTION

Nanopore sensor devices may typically employ an electrical signal across a nanopore channel to generate a measurement signal that is interpreted to sense and/or characterise molecular entities as they interact with the nanopore. Typically an electrical signal is applied as a potential difference or current across the array of sensor elements (also referred to as nanopore channels) that will provide a meaningful measurement signal to be interpreted. The measurement can include, for example, one of ionic current flow, electrical resistance, or voltage. Such nanopore sensor devices can provide long continuous reads of polymers, for example in the case of polynucleotides ranging from many hundreds to tens of thousands (and potentially more) nucleotides.

Generally speaking, such sophisticated sensor devices require powerful computers in order to analyse and interpret the signals generated by the sensor devices during their use. Due to the complexity of the generated signals and the potentially large amount of data being generated from the analysis complex analytes, it is desired to have a computer with enough processing capability and memory to carry out full analysis of the data generated by sensor devices. This is coupled with the desire to produce a device that is easily transportable and can be readily set up in a variety of conditions. Recent advances in semiconductor fabrication have provided tablet computers with a higher level of performance. Such tablet computers are able to process more complex operations and handle much bigger data sets than before.

In a first aspect, the present invention relates to a sensor device for use with a sensor component, the device comprising: a chassis and a lid, the lid being pivotally attached to the chassis and can be held in a closed position relative to the chassis, the chassis comprising: a recess portion on its upper surface for receiving the sensor component; and a retractable retaining feature, wherein when the lid is in the closed position at least part of the retaining feature is retracted into the chassis of the sensor device; and when the lid is moved from the closed position, the retaining feature moves to be proud from the recess portion.

The height profile of a sensor device that requires an insertable sensor component can be reduced by the incorporation of a retractable retaining feature on the chassis of the sensor device. This means that when the device is in an inactive mode without a sensor component inserted, the lid can be moved to a closed position and the device can be provided in a slimline state ready for transportation in, for example, a case for a tablet computer. However, the sensor device can be readily configured to be in an active state by moving the lid from the closed position such that the retractable retaining feature is proud of the upper surface of the chassis and ready to receive a sensor component for analysis.

In examples, when the lid is in the closed position, the majority of the retaining feature can be said to be substantially flush with the plane or surface of the recess portion to allow for the lid to close. In this connection, the chassis may be provided with a recess portion which may be shaped to receive the lid in the closed position such that the lid is flush with the outer surfaces of the chassis. In other words, when the sensor device is viewed side on, the lid appears to share the adjacent surfaces of the chassis so that a minimal profile can be achieved.

In addition to the closed position, the lid may be held in a raised position relative to the chassis, such that the retaining feature is able to hold a sensor component on the upper surface of the chassis when lid is held in the raised position. It is often desirable to shield the sensor component when the sensor device is in the active state such as when the sensor device is carrying out analysis of a sample. Thus, being able to hold the lid over the sensor component provides adequate shielding for when the sensor device is being used in analysis. However, due to the slim profile of the sensor device, it may not be possible to provide a lid which can fully enclose the sensor component when the lid is in the closed position.

The lid may be pivotally connected to the chassis such that the lid is substantially parallel to the chassis when the lid is held in the raised position. Furthermore, the lid may be pivotally connected to a hinge portion, and the hinge portion may be pivotally connected to the chassis such when the lid is in the closed portion, the hinge portion sits in the recess portion of the chassis and is flush with the outer surfaces of the chassis. In other words, the pivot points of the hinge enable it to lie flush with the chassis in a parallel plane to the recess portion, such that when the lid is in the closed position and the sensor device is viewed side on, the hinge portion and the pivotal connection of the lid and hinge portion appears to share the adjacent surfaces of the chassis so that a minimal profile can be achieved and the lid can also be held in a raised position parallel to the chassis.

The upper surface of the chassis may further comprise a plurality of electronic connections for communicating with a plurality of connectors located on a sensor component. These may also be provided in the recess portion, so that it may be designed such that the retaining feature functions merely to hold the sensor component in a particular position such that the sensor component can electronically communicate with another part or feature of the chassis. In this connection, the design of the retaining feature can be simplified significantly and does not require, for instance, an electronic interface with the sensor component. Furthermore, the electronics of the sensor device can be located away from the retractable retaining feature, such that it has enough space or volume in the chassis in which to retract when the lid is in the closed position. The electronic connections of the upper surface of the chassis are covered and protected by the lid when the lid is in the closed position.

The upper surface may comprise a slot for receiving at least part of the retaining feature when the sensor device is in the closed position. As such, the slot presents a void or space in the chassis in which at least part of the retaining feature can retract into when the lid is moved to the closed position. In examples, the retaining feature may comprise a clip attached to a stanchion part, such that at least the stanchion retracts in and out of the slot to move the clip from an inactive position (i.e. lid closed) to an active position (i.e. lid not closed), whereby in an active position the clip is ready to hold a sensor component on the upper surface (possibly in the recess portion) of the chassis.

The chassis may comprise means for releasably holding the at least part of the retaining feature within the chassis when the lid is in the closed position. The means for releasably holding the at least part of the retaining feature within the chassis may be at least one magnet positioned in the chassis. This ensures that the lid can be firmly shut when not in use to protect elements such as the electronic components, but potentially easily reopened when needed.

The lid may comprise a means for releasably engaging with at least part of the retaining feature such that when the lid is moved from the closed position, the at least part of the retaining feature is drawn through the slot to be proud of the recess. The means for releasably engaging with the retaining feature may be at least one magnet positioned on the lid. The movement of the lid causes the retaining feature to move from its retracted position since it is releasable coupled to at least part of the retaining feature. Thus, moving the lid from the closed position ensure that the sensor device is ready to receive a sensor component.

The chassis may comprise means for releasably securing at least part of the retaining feature, such that when the lid becomes disengaged from the retaining feature, the retaining feature is secured proud of the upper surface of the chassis. The means for releasably securing the at least part of the retaining feature within the chassis may be at least one magnet positioned in the chassis. The retaining feature can be resilient such that it can hold a sensor component in place when required. Securing the retaining feature in a proud position improves the ease of inserting and removing sensor components in the sensor device.

Alternatively, the chassis may comprise means for biasing the retaining feature into a proud position within the chassis when the lid is moved from the closed position. In such an embodiment, the lid may be held in a closed position by magnets and the retaining feature, or a part thereof, is biased against the lid, ready to move to a proud position when the lid is moved from a closed position. This ensures that the retaining feature is always in place ready to receive a sensor component. It may also provide a simplified design since the securing magnets previously described may not be required.

As mentioned previously, it is preferable that the lid is connected to the chassis such that the lid can be held in a raised position over a sensor component when a sensor component is held on the upper surface of the chassis. In the embodiments described above, the lid may rest on at least a part of the retaining feature when it is not retracted (preferably the stanchion of the retaining feature). In a particular embodiment, the lid may use the means for releasably engaging with the retaining feature to rest on in the raised position to provide a stable and level lid which can be released from that position with relative ease.

In a second aspect, the present invention provides a kit comprising the sensor device according to the statements above and a sensor component. The sensor component may comprise a nanopore array for receiving a sample to be analysed. Nanopore sensor devices require the handling of complex data signals and processing of information. However, the present invention provides a slimline device which can carry out these complex analysis operations.

The sensor component may comprise a plurality of connectors located on the underside of the component for connection with a respective plurality of connectors located at the upper surface of the sensor device.

In a third aspect, the present invention provides a sensor system comprising: a removable sensor device in accordance with the statements above; a hub component; and a tablet computer; the hub component is connectable to the sensor device, such that the hub component provides an interface for powering the sensor device and transferring data from the sensor device, and the hub component further comprises a connection to a tablet computer, such that the hub can deliver data collected from the sensor device to the tablet computer, wherein the height profile dimensions of the sensor device and the hub are similar to the height profile dimension of the tablet computer when the lid of the sensor device is in the closed position.

Previous attempts and designs for integrating a tablet computer directly with a nanopore device would require the full integration of a nanopore device into a case with a tablet (for example https://nanoporetech.com/products/minion-mk1d). However such a setup is undesired since it requires hard wiring a nanopore device into the case, meaning that the case manufacture becomes much more complex, and the servicing or replacement of parts is much harder. In addition, it is nor desirable to handle liquid samples, such as biological samples containing bacterial or viral DNA over or on the tablet computer or computer parts since if spilled the liquid would likely contaminate or damage to tablet computer or components of the case (for example an integrated keyboard). Furthermore, the integration of present sensor devices into cases adds unnecessary expense to an otherwise inexpensive article, and limits the user to a particular type of sensing. The removable sensor may be configurable to sense other analytes, materials, chemical moieties. Hence, the present invention also provides a standard setup potentially for use a variety of dockable sensors.

The hub component may further comprise an interfacing unit for connection to the tablet computer, wherein said interfacing unit comprises a USB-C interface for connecting to said tablet computer. The hub component may also comprise a USB-C power input, such that the hub component supplies power to both the sensor device and the tablet computer. The power delivered to the tablet computer may also be derived from the hub component via the USB-C interface of the interfacing unit. The above-mentioned details all allow for a simplified and elegant design which can be transported easily around a test environment without the requirement for a power lead or power source, since the power may be derived from the battery of the tablet computer, or a separate battery source plugged in to the system via, for example, a USB-C interface.

The sensor device, the hub component and the tablet computer may all be provided in a case. Thus, the sensor device can be transported and used in a variety of test environments and be stored safely when not in use. The case may optionally be divided into two segments. One larger segment would be used to house or hold the tablet computer and possibly at least a part of a hub component. A smaller segment would be used to house the sensor device and at least part of the hub component. The case may optionally include a lid portion that would cover the tablet computer and or sensor device when not in use.

An element may be provided in the case which is foldable such that the tablet computer can be configured into a flat position or into a raised position. This may be provided as a rigid set of components in the lid support the tablet computer and or sensor device when in the raised position. Alternatively, this may for example take the form of a pair of legs which can support the case when the tablet computer is configured in a raised position, the legs being stowed flush within the case when the tablet computer is configured in the flat position. The legs may be stowed in the segment supporting the sensor device. The legs attach to the case via a cam and hinge so as to allow the segment housing the tablet computer to be held in the raised position.

The case may comprise a segment for housing the sensor device, and the case is configured such that when the tablet computer is in either position, the sensor device is maintained in a flat position, relative to the surface on which the case rests. In essence, the segment housing the sensor device can be maintained in a parallel position to the surface on which the case rests, such as a lab bench or work bench. The tablet computer and the sensor device can be allowed to cool once in the raised position due to surface(s) exposure to air (rather than being support against the work bench in the flat position).

In this connection, the case may further comprise a segment for housing the tablet computer which incorporates a duct for cooling the tablet computer. The segment for housing the sensor device may also comprise a fan which is in fluid communication with the duct. This allows for the passage of air against the surface of the tablet computer to aid in cooling of the tablet computer when it is engaged in activities requiring a large amount of processing power (for example determining the sequence a biological sample via the sensor device).

The sensor device of the sensor system may further comprise a removable sensor component, and sensor component may comprise a nanopore array for receiving a sample to be analysed.

### DESCRIPTION OF THE FIGURES

To allow better understanding, embodiments of the present invention will now be described by way of non-limitative example with reference to the accompanying drawings, in which:

FIG. 1 is a simplified isometric view of a sensor device in accordance with the present invention with the lid in a closed position;

FIG. 2 is a simplified isometric view of a sensor device in accordance with the present invention with the lid in a raised position;

FIG. 3 is a simplified isometric view of a sensor device in accordance with the present invention and a sensor component wherein the lid of the sensor device is shown in an open position;

FIG. 4 is an isometric view of a sensor device in accordance with the present invention and a sensor component wherein the lid of the sensor device is shown in an open position and the sensor component is inserted in the sensor device;

FIGS. 5a-d are side cross-sectional views of the sensor device in accordance with the present invention in various configurations with the chassis bar the upper surface removed;

FIG. 6 is a top view of the sensor device in accordance with the present invention whereby the lid is transparent;

FIGS. 7a-b are isometric views of the sensor system of the present invention, wherein the sensor device is shown to be removable;

FIGS. 8 and 9 are isometric views of an alternative case arrangement for the sensor device and tablet computer shown in flat and raised configurations, respectively;

FIG. 10 is an isometric view of a segment of the case for supporting the sensor device displaying the hinge arrangement; and

FIG. 11 is a plan view of a segment of the case for supporting the sensor device displaying the hinge arrangement.

The various features described below are examples and not limitative. Also, the features described are not necessarily applied together and may be applied in any combination.

### DETAILED DESCRIPTION

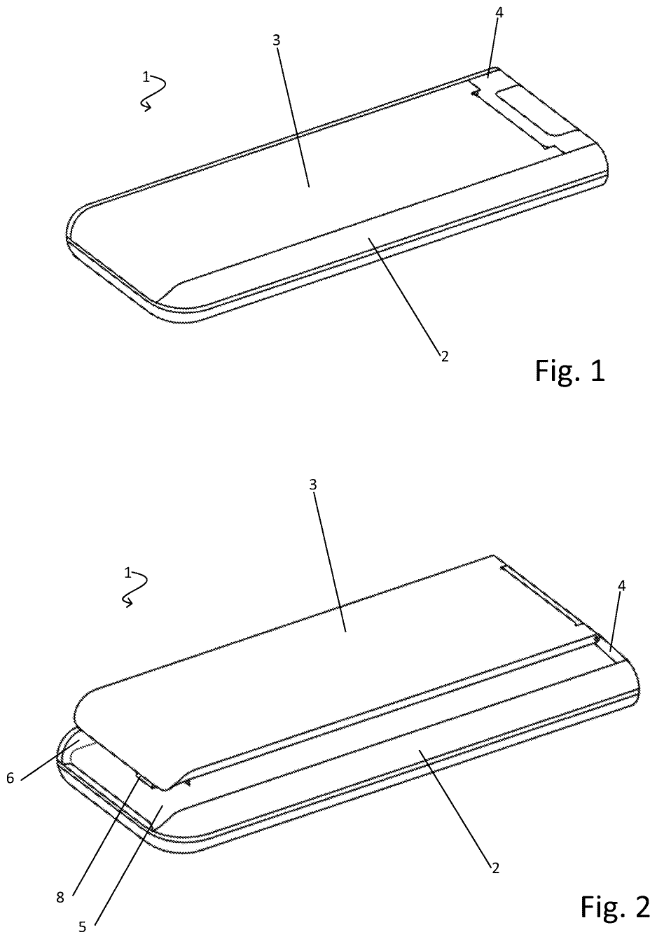

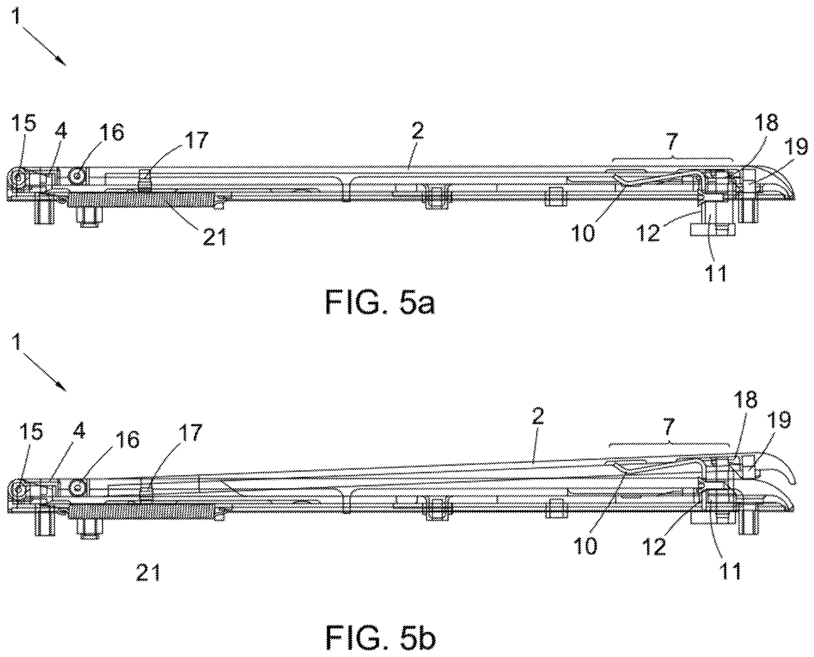

As shown in FIGS. 1-4, the sensor device 1 comprises a chassis 2, a lid 3, a hinge portion 4. The chassis 2 comprises an upper surface 5 which comprises a recess portion 6 which is revealed when the lid 3 is moved from a closed position. The recess portion 6 is shaped so as to receive the lid 3 when it is in the closed position. As can be seen in FIG. 1, the recess portion 6 accommodates the lid 3 such that it is flush and within the footprint of the chassis 2 when the lid 3 is in the closed position.

The upper surface 5 comprises a retaining feature 7 for retaining and holding a sensor component 8 during use of the sensor device 1. The upper surface 5 also includes an electrical interface 9 for connection and communication with a sensor component 8. The electrical interface 9 comprises a plurality of a plurality of electronic connections for communicating with a plurality of connectors located on a sensor component 8. Thus the sensor device 1 can communicate with a plurality of channels and or sensors on the sensor component 8 when inserted.

A suitable sensor component 8 includes but is not limited to a disposable or reusable test cartridge, such as point of care diagnostic device, a nanopore array or an analyte detector, such as a glucose sensor.

The retaining feature 7 comprises a clip 10 mounted to a stanchion 11. The stanchion 11 and part of the clip 10 are movable within a slot 12 located in the chassis 2, forming part of the upper surface 5 of the recess portion 6. The movement and function of the retaining feature 8 will be described in more detail later.

The chassis 2 also comprises a case portion 13 with a series of LEDs 14 for communicating with the user during use of the sensor device 1. The case portion 13 is surrounded by the hinge portion 4 so that it is visible in whatever position the lid 3 is presented in. In this connection, the hinge portion 4 has a cut-away section to allow for the user to view the LEDs 14, even if the lid 3 is in the closed position (i.e. the LEDs 14 are not obscured by the lid 3, even when in a closed position).

The hinge portion 4 is pivotally connected to the chassis 2 via a rotatable hinge 15. Similarly, the hinge portion 4 is pivotally connected to the lid 3 via a rotatable hinge 16. The dual pivotal connectors 15,16 of the hinge portion 4 to the chassis 2 and the lid 3, respectively, allows for full articulation of the lid 3 relative to the chassis 2. This will be described in more detail below.

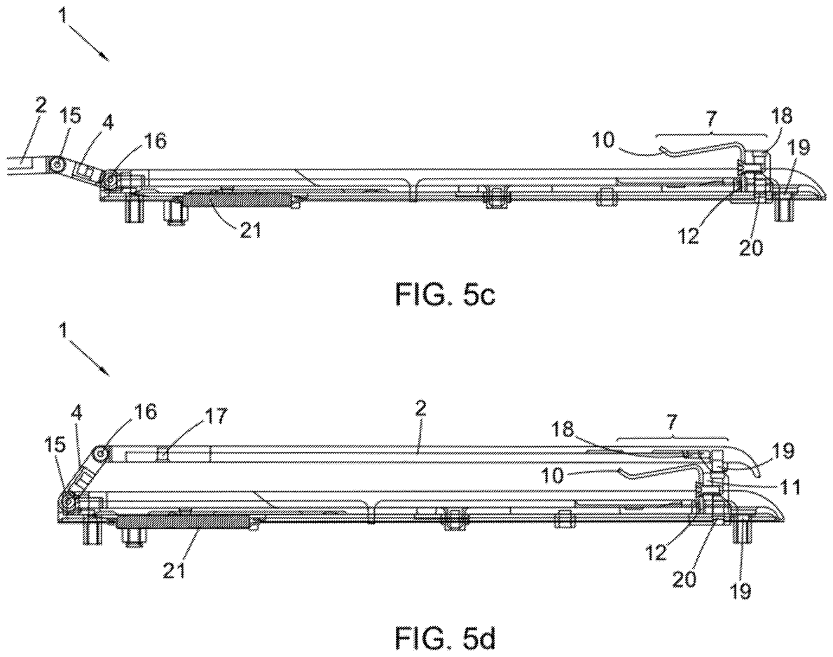

FIGS. 5a-d illustrate the movement of the lid 3 relative to the chassis 2 from a closed position (FIG. 5a) to a partially open position (FIG. 5b), to a fully extended position (FIG. 5c) and a raised position (FIG. 5d).

In FIG. 5a the lid 3 is provided in a closed position (akin to the embodiment shown FIG. 1). The lid 3 and the hinge portion 4 are flush with the chassis 2 (not shown). In addition, the pivotal connector 16 connecting the lid 3 to the hinge portion 4 is located within the footprint of the chassis (i.e. within the recess portion 5 of the chassis 2).

The lid 3 is releasably held in place in the recess portion 5 of the chassis 2 by several pairs of magnets 17,18,19 (simplified in some Figures to identify only one of each of the pair of magnets).

The lid 3 has three magnets 17,18,19 which, in the closed position, correspond to magnets 17,19 located on the chassis, and a magnet 18 located on the stanchion 11 of the retaining feature 8. The lid 3 is held firmly in place in the closed position by the pairs of magnets 17,18,19. Furthermore, the pair of magnets 18 located on the lid 3 and the stanchion 11 of the retaining feature 8 are releasably engaged, such that on lifting the lid 3, the magnets 17 remaining in contact and move the retaining feature 8 out of the slot 12 located on the chassis 2. The magnets 17,19 on the lid 3 and chassis 2 may be weaker relative to the magnets 18 on the lid 3 and retaining feature 8. Alternatively, the retaining feature 8 may be biased toward the lid 3 such that the appropriate magnets 17 maintain contact with each other as the lid 3 is moved from the closed position (see FIG. 5b). In any event, once the lid 3 is moved from the closed position, the retaining feature 8 is caused to move away from the upper surface 5 of the chassis 2.

The movement of the retaining feature 7 is such that clip 10 and stanchion 11 travel together along the vertical axis of the stanchion 11. In this embodiment, the clip 10 is affixed to the stanchion 11, although the clip 10 and stanchion 11 may be manufactured as one individual part. The slot 12 is shaped such that it can receive the stanchion 11 and part of the clip 10. The motion of the retaining feature 7 is a simple motion along particular axis (i.e. up and down) so as to provide minimal complexity to the sensor device 1. The stanchion 11 is guided by the edges of the slot 12 and magnets 18,20 can be affixed to it for supporting the lid so that the clip 10 (and potentially the sensitive sensor component) is not interfered with during use or opening/closing of the lid 3.

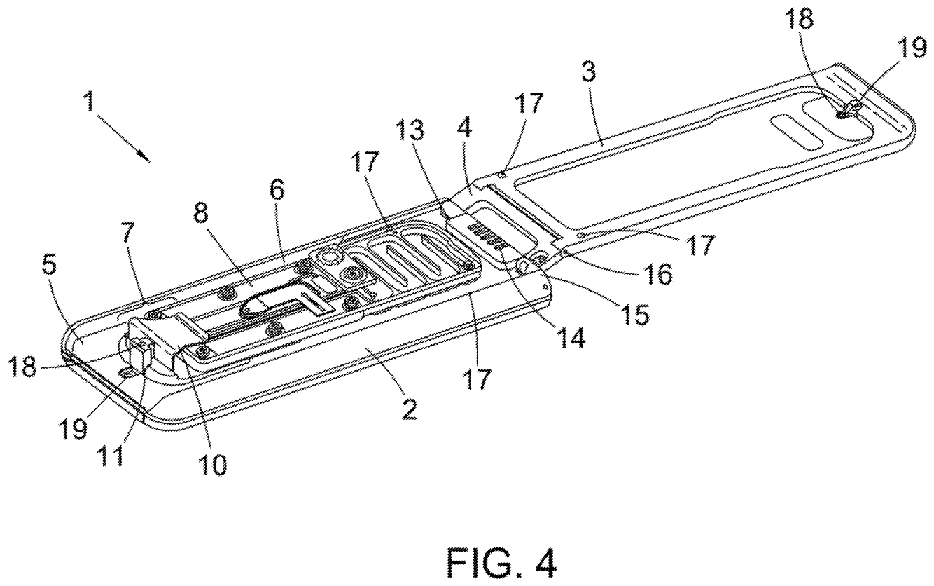

As shown in FIGS. 3 and 5c, once the lid 3 has moved a position wherein the pair of magnets 18 on the lid 3 and stanchion 11 are no longer in contact, the retaining feature 8 is proud of the upper surface 5 of the chassis 2. This enables a sensor component 8 to be inserted into the sensor device 1 when the lid 3 is in, for example, a prone-type position as shown in FIG. 5c. The lid 3 is biased from fully extending.A spring 20 is used to restrict the lid 2 moving beyond a particular position. This can be useful in event that the sensor device 1 is adjacent to other testing accessories, or it is desirable for the sensor device 1 to not become, for example, contaminated or damaged by contact other surfaces.

At this point, the retaining feature is secured in its position by another pair of magnets 20 located within the chassis 2. However, as stated above, the retaining feature 6 may also be secured in this position by biasing means (not shown).

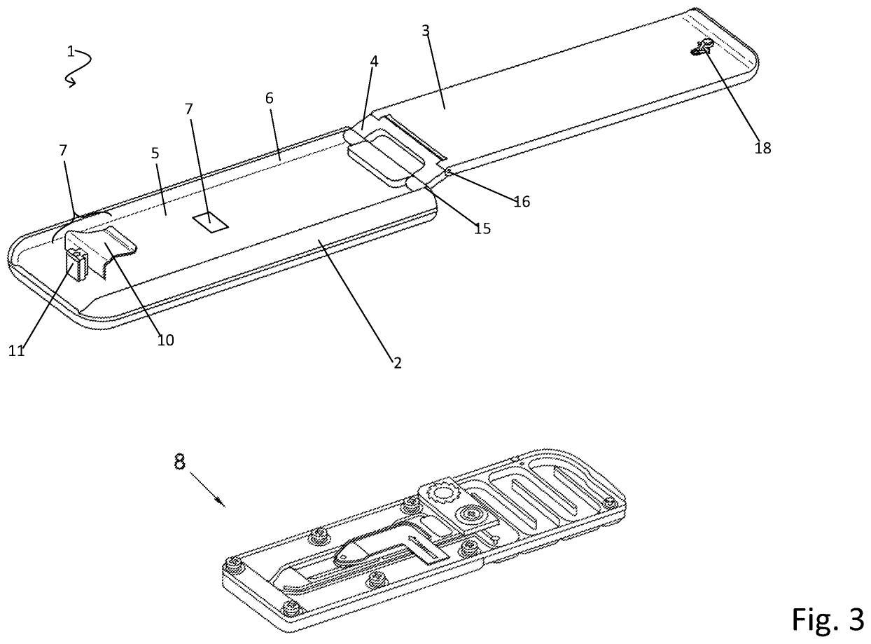

The lid 3 can be adopted and rested into a raised position (shown in FIGS. 2 and 5d). In use, the recess portion 6 of the upper surface 5 would contain a sensor component 8. The lid 3 would partially enclose the sensor component 8 so that it is sufficiently shielding, for example during the analysis of a sample. In this particular embodiment, the pairs of magnets 18,19 provided on the lid contact such that the corresponding magnet 19 on the lid 3 contacts the magnet 16 on the stanchion 9. The geometry of the lid 3 and the polarity of the pairs of magnets 18,19 is set out such that the lid 3 is able to rest in this position. The spring 20 is under tension such that the hinge portion 4 can rest, allowing for the lid 3 to remain substantially parallel to the upper surface 5 of the chassis 2.

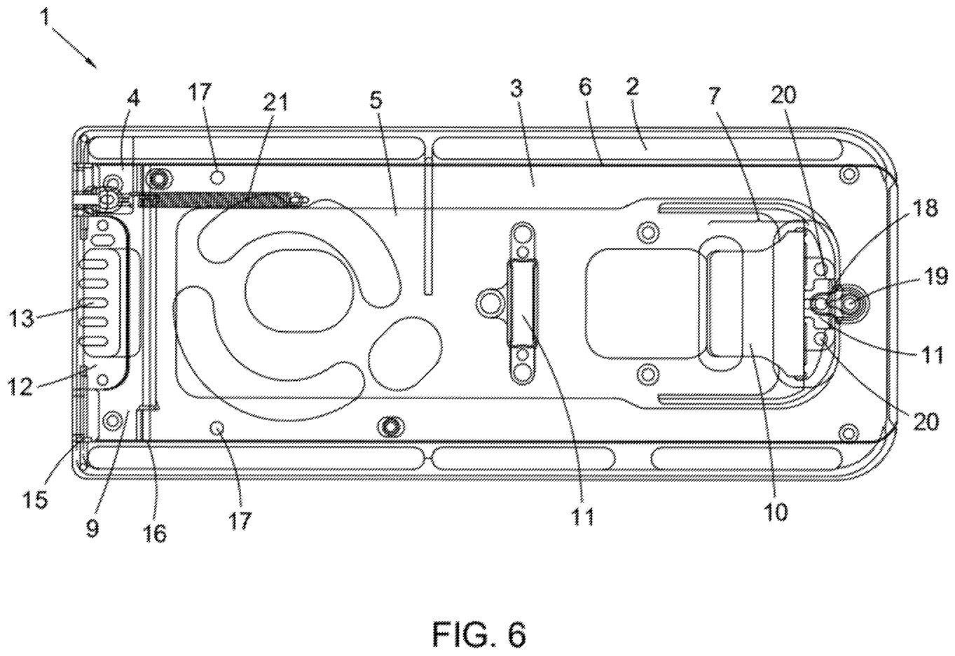

FIG. 6 shows a top view of the sensor device 1 with a layout of the pairs of magnets 17,18,19. The lid 3 has been made translucent so as not to obscure the relevant parts.

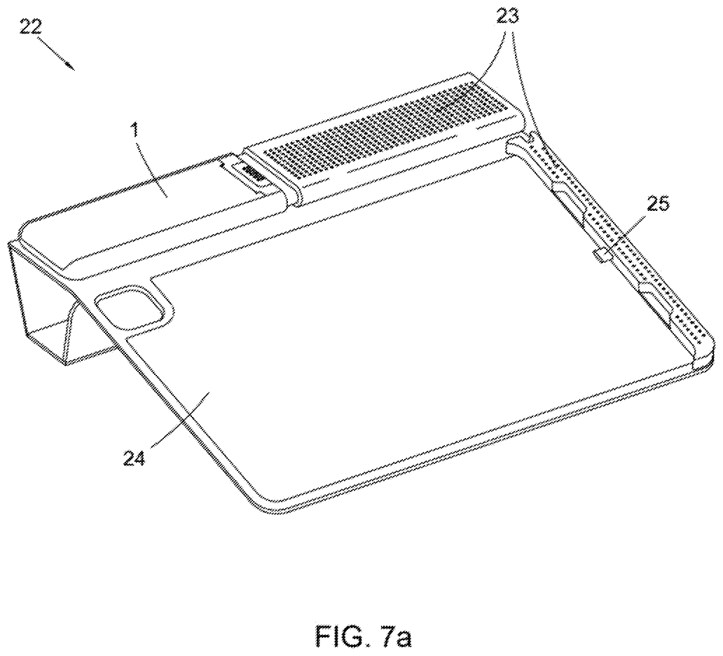

FIGS. 7a and 7b show a sensor system 22 comprising a removable sensor device 1 according to the statements above. The sensor system 22 also comprises a hub component 23; and a tablet computer (not shown). The sensor device 1, the hub component 23 and the tablet computer (not shown) are all provided in an example of a case 24.

In FIG. 7b, the sensor system 22 is shown where the sensor device 1 has been removed, for example, to prepare for analysis away from items that might be sensitive to liquid (i.e. the tablet computer or the hub component 23).

The hub component 23 is connectable to the sensor device 1, such that the hub component 23 provides an interface for powering the sensor device 1 and transferring data from the sensor device 1 to a tablet computer (not shown), and the hub component 23 further comprises a connection to the tablet computer (not shown), such that the hub component 23 can deliver data collected from the sensor device 1 to the tablet computer (not shown).

As can be seen FIG. 7a, the height profile dimensions of the sensor device 1 and the hub component 23 are similar to the height profile dimension of a tablet computer (not shown) when the lid of the sensor device 1 is in the closed position. The hub component 23 further comprises an interfacing unit for connection to the tablet computer. The interfacing unit comprises a USB-C interface 25 for ease of connection to the tablet computer (not shown). The hub component 23 comprises a USB-C power input for ease of connection with standard power supplies. The hub component 23 is able to supply power to both the sensor device 1 and the tablet computer (not shown) whilst transferring data. Ideally the power delivered to the tablet computer (not shown) from the hub component 23 is via the USB-C interface 25 of the interfacing unit 23.

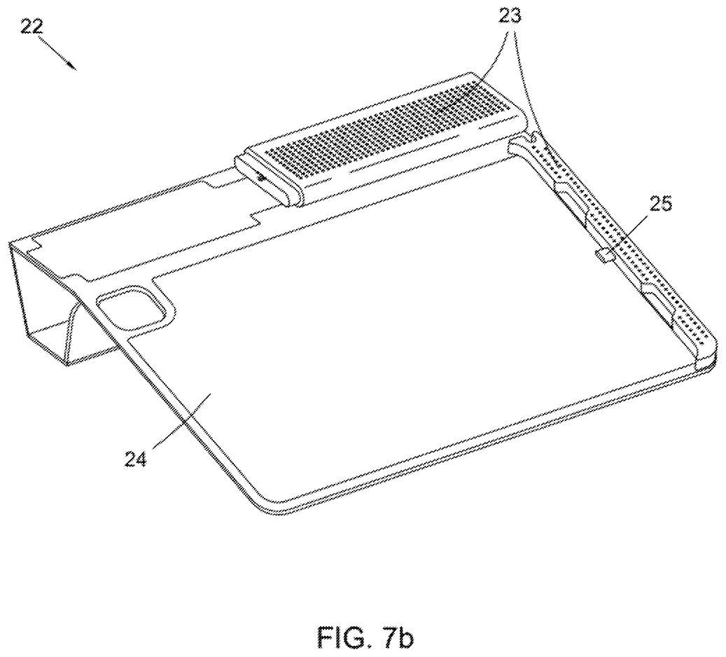

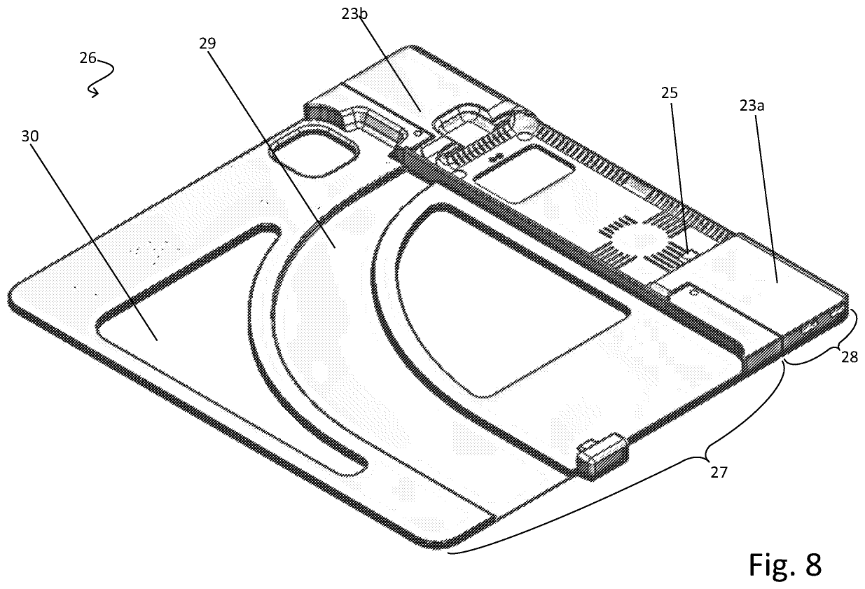

FIGS. 8 to 11 show an alternative case arrangement 26 which also can be configured such that the tablet computer (not shown) can be held in both a flat position (see FIG. 8) and raised position (see FIG. 9) relative to a worktop or surface. The alternative case arrangement 26 is divided into two segments 27,28 for housing a tablet computer (not shown) and a sensor device (not shown).

The segment 28 for housing or holding the sensor device 1 comprises the hub components 23a,23b which in this alternative case arrangement 26 is split in two. One hub component 23a relies on a USB-C interface 25 to communicate to the sensor device 1. The second hub component 23b houses a fan for cooling the sensor device 1 and or the tablet computer (not shown).

The fan in the second hub component 23b is in fluid communication with a duct 29 in the segment 27 for housing the tablet computer (not shown). The duct 29 allows air to pass over the tablet computer (not shown) when in the system is in use. The duct 29 may act passively, or may act to channel air or fluid over a surface of the tablet computer when the fan in the second hub component 23b is operational. Furthermore, cut outs 30 in the segment 27 housing the tablet computer can be provided.

Cooling is expected to be more efficient when the tablet computer (not shown) is provided in a case 24,26 in the raised position (see FIG. 7a andb, and FIG. 9). In addition, perceived usability of the device will also be improved in this position, for example screen glare and readability of the screen on the table computer (not shown).

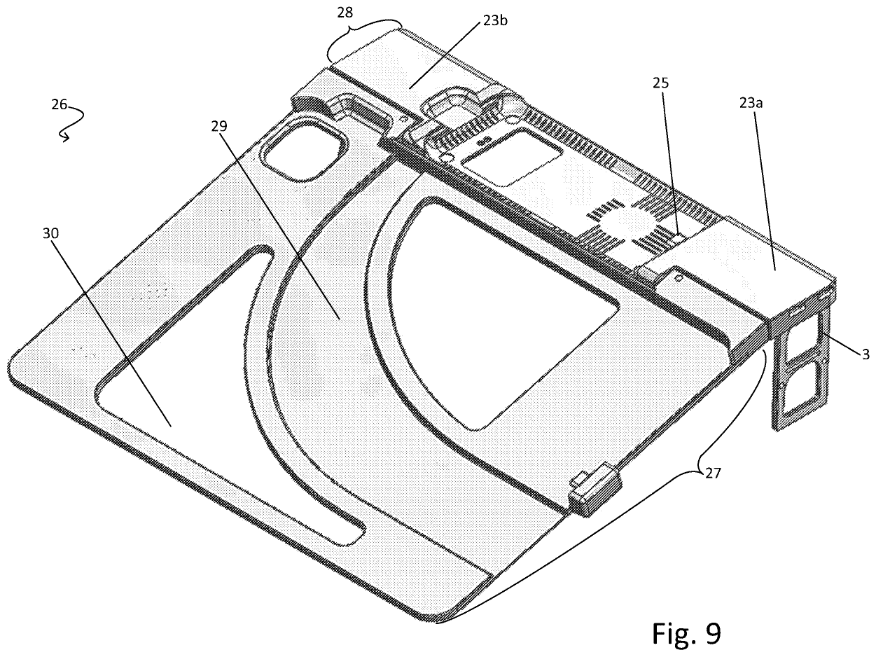

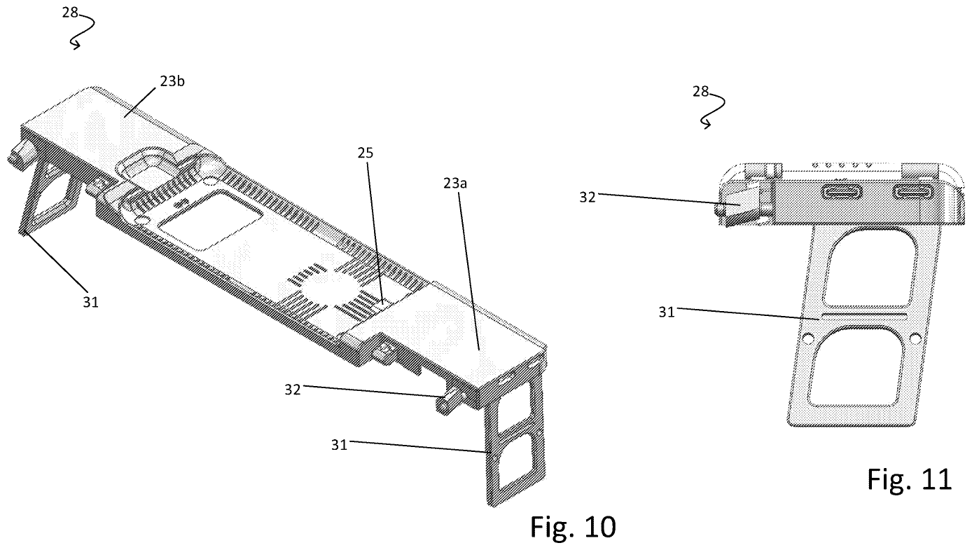

FIGS. 10 and 11 show an arrangement of elements 31 that are used to maintain the alternative case arrangement 26 in a configuration where the tablet computer (not shown) is in a raised position. As the elements 31 fold out from a stowed position from onboard the segment 28 for housing or holding the sensor device 1, they cause a cam 32 within the segment 27 for housing or holding the tablet computer (not shown) to rotate and rest such that the segment 27 for housing or holding the tablet computer (not shown) can rest such that the tablet computer (not shown) rests in a raised position. When required, the elements 31 can be folded back to a stowed position onboard the segment 28 for housing or holding the sensor device 1 such that they are flush within the segment 28. The whole alternative case arrangement 26 can now lie flat on a surface so that, for example, a sample can be more stably loaded or prepared in the sensor component 8 within the sensor device 1.

1. A sensor device for use with a sensor component, the device comprising:
a chassis and a lid, the lid being pivotally attached to the chassis and which can be held in a closed position relative to the chassis,
the chassis comprising: an upper surface for receiving the sensor component; and
a retractable retaining feature for holding the sensor component in position on the upper surface,
wherein when the lid is in the closed position at least part of the retaining feature is retracted into the chassis of the sensor device; and when the lid is moved from the closed position, the retaining feature moves to be proud from the upper surface.

2. The sensor device according to claim 1, wherein the upper surface comprises a recess portion, the recess portion being shaped to receive the lid in the closed position such that the lid is flush with the outer surfaces of the chassis.

3. The sensor device according to claim 1 or claim 2, wherein the lid can be held in a raised position relative to the chassis, such that the retaining feature is able to hold the sensor component on the upper surface when lid is held in the raised position.

4. The sensor device according to claim 3, wherein the lid is pivotally connected to the chassis such that the lid is substantially parallel to the chassis when the lid is held in the raised position.

5. The sensor device according to any one of claims 2 to 4, wherein the lid is pivotally connected to a hinge portion, and the hinge portion is pivotally connected to the chassis such when the lid is in the closed portion, the hinge portion sits in the recess portion of the upper portion and is flush with the outer surfaces of the chassis.

6. The sensor device according to any one of the previous claims, wherein the upper surface further comprises a plurality of electronic connections for communicating with a plurality of connectors located on a sensor component.

7. The sensor device according to any one of the previous claims, wherein the upper surface comprises a slot for receiving at least part of the retaining feature when the sensor device is in the closed position.

8. The sensor device according to claim 7, wherein the chassis comprising means for releasably holding the at least part of the retaining feature within the chassis when the lid is in the closed position.

9. The sensor device according to claim 7, wherein the chassis comprising means for biasing the retaining feature into a proud position when the lid is moved from the closed position.

10. The sensor device according to any one of claims 1 to 8, wherein the lid comprises a means for releasably engaging with at least part of the retaining feature such that when the lid is moved from the closed position, the at least part of the retaining feature is drawn through the slot to be proud of the upper surface.

11. The sensor device according to claims 10, wherein the chassis comprises means for releasably securing at least part of the retaining feature, such that when the lid becomes disengaged from the retaining feature, the retaining feature is secured proud of the upper surface.

12. The sensor device according to any one of claims 8, wherein the means for releasably holding the at least part of the retaining feature within the chassis is at least one magnet positioned in the chassis.

13. The sensor device according to any one of claims 10, wherein the means for releasably engaging with the retaining feature is at least one magnet positioned on the lid.

14. The sensor device according to claims 11, wherein the means for releasably securing the at least part of the retaining feature within the chassis is at least one magnet positioned in the chassis.

15. A kit comprising the sensor device according to any one of claims 1 to 14, and a sensor component.

16. The kit according to claim 15, wherein the sensor component comprises a nanopore array for analysing a sample.

17. The kit according to claim 16, wherein the sensor component comprises a plurality of connectors located on the underside of the component for connection with a respective plurality of connectors located at the upper surface of the sensor device.

18. A sensor system comprising:
a removable sensor device according to any one of claims 1 to 14;
a hub component; and
a tablet computer;
the hub component is connectable to the sensor device, such that the hub component provides an interface for powering the sensor device and transferring data from the sensor device, and the hub component further comprises a connection to the tablet computer, such that the hub can deliver data collected from the sensor device to the tablet computer,
wherein the height profile dimensions of the sensor device and the hub are similar to the height profile dimension of the tablet computer when the lid of the sensor device is in the closed position.

19. The sensor system according to claim 18, wherein the hub component further comprises an interfacing unit for connection to the tablet computer, wherein said interfacing unit comprises a USB-C interface for connecting to said tablet computer.

20. The sensor system according to claim 19, wherein the hub component comprises a USB-C power input, such that the hub component supplies power to both the sensor device and the tablet computer.

21. The sensor system according to claim 20, wherein the power delivered to the tablet computer from the hub component is via the USB-C interface of the interfacing unit.

22. The sensor system according to according to any one of claims 18 to 21, wherein the sensor device, the hub component and the tablet computer are all provided in a case.

23. The sensor system according to according to claim 22, wherein an element in the case is foldable such that the tablet computer can be configured into a flat position or into a raised position.

24. The sensor system according to according to claim 23, wherein the case comprises a segment for housing the sensor device, and the case is configured such that when the tablet computer is in either position, the sensor device is maintained in a flat position.

25. The sensor system according to according to claim 23, wherein the case further comprises a segment for housing the tablet computer which incorporates a duct for cooling the tablet computer.

26. The sensor system according to claim 25, wherein the segment for housing the sensor device comprises a fan which is in fluid communication with the duct.

27. The sensor system according to according to any one of claims 23 to 26, wherein the element is a pair of legs which can support the case when the tablet computer is configured in a raised position, the legs being stowed flush within the case when the tablet computer is configured in the flat position.

28. The sensor system according to any one pf previous claims 22 to 27, wherein the sensor device further comprises a sensor component.

29. The sensor system according to claim 28, wherein the sensor component comprises a nanopore array for receiving a sample to be analysed.
